(19) United States
(12) Patent Application Publication
(10) Pub. No.: US 2025/0032656 A1
(43) Pub. Date: Jan. 30, 2025

(54) UV DISINFECTION ELECTRONIC DEVICE AND PROCESSING METHOD

(71) Applicant: WISTRON CORPORATION, New Taipei City (TW)

(72) Inventors: Lipin CHIANG, New Taipei City (TW); Chien En PENG, New Taipei City (TW)

(73) Assignee: WISTRON CORPORATION, New Taipei City (TW)

(21) Appl. No.: 18/536,991

(22) Filed: Dec. 12, 2023

(30) Foreign Application Priority Data
Jul. 27, 2023 (TW) 112128246

(51) Int. Cl. A61L 2/24; A61L 2/10; A61L 2/26

(52) U.S. Cl. CPC A61L 2/24; A61L 2/10; A61L 2/26; A61L 2202/11; A61L 2202/14

(57) ABSTRACT

An Ultraviolet (UV) disinfection electronic device includes a mobile device, a UV lamp group, and a microcontroller. The UV lamp group, and the microcontroller are disposed at the mobile device. The UV lamp group includes multiple UV lamps and a driving circuit. The microcontroller is connected to the autonomous mobile robot and the driving circuit. The driving circuit is connected to the UV lamp group and the microcontroller. The driving circuit is used to drive the UV lamps. The microcontroller, in response to an activation command, activating the mobile device to move at a travel speed of a speed requirement and the driving circuit to drive a number of UV lamps to emit an UV light. The number is related to the speed requirement and disinfection need.

### CROSS-REFERENCE TO RELATED APPLICATION

This application claims the priority and benefit of Taiwan Patent Application No. 112128246, filed on Jul. 27, 2023, the disclosure of which is hereby incorporated in its entirety by reference herein.

### TECHNICAL FIELD

This case relates to an electronic device and a processing method thereof, and in particular to an Ultraviolet (UV) disinfection electronic device and a processing method.

### RELATED ART

Ultraviolet C (UVC) is a disinfection option with low cost, wide range and high efficiency. Typical UV disinfection devices are based on UVC. UVC, because of its wavelength range of 280 nm to 100 nm, can destroy structures of deoxyribonucleic acid (DNA) and ribonucleic acid (RNA) of organisms, thus inhibiting self-replication of organisms. UVC has the same effect on microbes such as viruses or bacteria, so it can also achieve the purpose of disinfection.

The irradiation dose of UVC may affect the sterilization ability. The irradiation dose is determined based on an irradiation energy (or radiant energy) and an irradiation time of UVC lamps. A too short irradiation time of the disinfection device on the target or a too low intensity of UVC will result in an insufficient irradiation dose, which cannot effectively inhibit the replication speed of viruses (or bacteria).

UVC may also do harm to the human body. Therefore, when the UV disinfection device is in operation, the user is often advised to leave the spatial field to which the target belongs. Moreover, the UVC lamps will become decayed over time, which will also reduce the irradiation dose. However, it is difficult for the user to adjust an UV light of the UVC lamps when the lamps have been decayed or damaged.

### SUMMARY

In view of this, this case provides an UV (Ultraviolet) disinfection electronic device and a processing method, which can automatically adjust an irradiation intensity of corresponding UV light according to a set travel speed such that the disinfection operation in an unmanned environment can meet the expected disinfection need.

In some embodiments, provided is an UV disinfection electronic device, applicable to an autonomous mobile robot. The UV disinfection electronic device includes a UV lamp group and a microcontroller. The UV lamp group is disposed on the autonomous mobile robot. The UV lamp group includes a plurality of UV lamps and a driving circuit. The driving circuit is electrically connected to the plurality of UV lamps, disposed on the autonomous mobile robot and configured to drive the plurality of UV lamps. The microcontroller is electrically connected to the autonomous mobile robot and the driving circuit. The microcontroller controls a travel speed of the autonomous mobile robot. The microcontroller drives the driving circuit according to a disinfection need to drive a number of UV lamps in the plurality of UV lamps to emit an UV light. The number of UV lamps is related to the travel speed and the disinfection need. The microcontroller adjusts the travel speed with respect to brightness of the UV light.

In this way, the UV disinfection electronic device may automatically select the corresponding number of UV lamps according to the set movement speed, such that the disinfection operation can meet the expected disinfection need.

In some embodiments, the UV disinfection electronic device further includes a plurality of light sensors. The plurality of light sensors are connected to the microcontroller and respectively correspond to the plurality of UV lamps. Each of the light sensors is disposed on a periphery of the corresponding UV lamp. Each of the light sensors is configured to acquire a photoinduced voltage of the corresponding UV lamp.

In some embodiments, the microcontroller is further configured to receive the photoinduced voltage of each of the UV lamps emitting the UV light, obtain an irradiation dose according to each of the photoinduced voltages and the travel speed corresponding to a speed requirement, and reduce the travel speed or increase the number of UV lamps according to the irradiation dose and the disinfection need in a case that the irradiation dose is relatively smaller than the disinfection need.

In some embodiments, the UV disinfection electronic device further includes a communication apparatus. The microcontroller is connected to the communication apparatus, and in a case that the irradiation dose is relatively smaller than the disinfection need, the microcontroller generates a notification message, and transmits the notification message to a monitoring platform or a client via the communication apparatus.

In some embodiments, the microcontroller is further configured to periodically receive and record the photoinduced voltage of each of the UV lamps emitting the UV light, and compare the photoinduced voltages of the same UV lamp at adjacent two receptions, and in a case that the photoinduced voltage of any of the UV lamps is relatively reduced, the microcontroller reduces the travel speed or increase the number of UV lamps.

In some embodiments, the UV disinfection electronic device further includes a communication apparatus. The microcontroller is connected to the communication apparatus, and in a case that the photoinduced voltage of any of the UV lamps is relatively reduced, the microcontroller generates a notification message, and transmits the notification message to a monitoring platform or a client via the communication apparatus.

In some embodiments, in a case that a speed requirement is relatively smaller than or equal to a threshold speed corresponding to the disinfection need, the microcontroller selects a speed option. The speed option is that the travel speed is set to the speed requirement or closest to and greater than the speed requirement.

In some embodiments, the UV disinfection electronic device further includes a communication apparatus. The microcontroller is connected to the communication apparatus, and in a case that the speed requirement is relatively greater than the threshold speed corresponding to the disinfection need, the microcontroller generates a notification message, and transmits the notification message to a monitoring platform or a client via the communication apparatus.

In some embodiments, in a case that the speed requirement is relatively greater than the threshold speed corresponding to the disinfection need, the microcontroller is further configured to set the travel speed to the threshold speed and determine that the number of UV lamps is a total number of the plurality of UV lamps.

In some embodiments, the UV disinfection electronic device further includes a communication apparatus. The microcontroller is connected to the communication apparatus. The microcontroller is further configured to accumulate an operation time of each of the UV lamps emitting the UV light, and in a case that the operation time of any of the UV lamps is relatively greater than or equal to a threshold time, the microcontroller generates a warning notification, and transmits the warning notification to a monitoring platform or a client via the communication apparatus.

In some embodiments, the UV disinfection electronic device further includes a communication apparatus. The microcontroller is connected to the communication apparatus. The microcontroller is further configured to receive a disinfection signal from a monitoring platform or a client via the communication apparatus. The disinfection signal includes an activation command.

In some embodiments, the disinfection signal further includes at least one of a need requirement of the disinfection need and the speed requirement.

In some embodiments, provided is an UV disinfection processing method, including: receiving, by an electronic device, a disinfection signal via a network, the disinfection signal including an activation command; determining, by the electronic device, a number of UV lamps according to a disinfection need and a speed requirement; selecting, by the electronic device, the number of UV lamps from a plurality of UV lamps of a UV disinfection device as an operating lamp; and activating, by the electronic device, the UV disinfection device in response to the activation command, enabling the UV disinfection device to advance at a travel speed corresponding to the speed requirement, and enabling each of the operating lamps to emit an UV light.

In some embodiments, the UV disinfection processing method further includes: receiving a photoinduced voltage of each of the operating lamps emitting the UV light; calculating an irradiation dose according to the photoinduced voltage of each of the operating lamps and the travel speed; comparing the irradiation dose with the disinfection need; and reducing the travel speed or increasing the number of UV lamps according to the irradiation dose and the disinfection need by the electronic device in a case that the irradiation dose is relatively smaller than the disinfection need.

In some embodiments, the UV disinfection processing method further includes: generating, by the electronic device, a notification message in a case that the irradiation dose is relatively smaller than the disinfection need; and transmitting the notification message to a monitoring platform or a client via a network.

In some embodiments, the UV disinfection processing method further includes: periodically receiving and recording a photoinduced voltage of each of the operating lamps emitting the UV light; comparing the photoinduced voltages of the same operating lamp at adjacent two receptions; and reducing the travel speed or increasing the number of UV lamps in a case that the photoinduced voltage of any of the operating lamps is reduced.

In some embodiments, the UV disinfection processing method further includes: generating, by the electronic device, a notification message in a case that the photoinduced voltage of any of the operating lamps is relatively reduced; and transmitting the notification message to a monitoring platform or a client via a network.

In some embodiments, the UV disinfection processing method further includes: confirming the speed requirement according to a threshold speed corresponding to the disinfection need, different disinfection needs corresponding to different threshold speeds; generating, by the electronic device, a notification message in a case that the speed requirement is relatively greater than the threshold speed; and transmitting the notification message to a monitoring platform or a client via a network.

In some embodiments, the UV disinfection processing method further includes: selecting, by the electronic device, a speed option in a case that the speed requirement is relatively smaller than or equal to the threshold speed, the speed option is that the travel speed is set to the speed requirement or closest to and greater than the speed requirement.

In some embodiments, in a case that the speed requirement is relatively greater than the threshold speed, in the step of determining the number of UV lamps according to the disinfection need and the speed requirement, the number of UV lamps determined is a total number of the plurality of UV lamps, and the processing method further includes: setting the travel speed to the threshold speed.

In some embodiments, the UV disinfection processing method further includes: accumulating an operation time of each of the UV lamps emitting the UV light; comparing the operation time of each of the UV lamps with a threshold time; generating, by the electronic device, a warning notification in a case that the operation time of any of the UV lamps is relatively greater than or equal to the threshold time; and transmitting the warning notification to a monitoring platform or a client via a network.

In some embodiments, the disinfection signal further includes at least one of a need requirement of the disinfection need and the speed requirement.

Based on the above, the UV disinfection electronic device and processing method according to any embodiment is applied to disinfection of a spatial field, and can adjust the number of UV lamps emitting the UV according to the travel speed and the disinfection need. In some embodiments, the UV disinfection electronic device or UV disinfection processing method can automatically adjust the travel speed or the number of UV lamps emitting the UV light when the illumination of any of the UV lamps is decayed. In some embodiments, the UV disinfection electronic device or UV disinfection processing method can notify a remote manager when adjusting the travel speed or the number of UV lamps. In some embodiments, the UV disinfection electronic device or UV disinfection processing method can record the operation time of any of the UV lamps, and notify the remote manager when the operation time of any of the UV lamps exceeds the threshold time. In some embodiments, the UV disinfection electronic device or UV disinfection processing method can notify the remote manager when the speed requirement is greater than the threshold time corresponding to the disinfection need, so as to reset the speed requirement and provide other speed requirements.

### BRIEF DESCRIPTION OF THE DRAWINGS

FIG. 1 is a schematic diagram showing architecture of an UV disinfection electronic device according to an embodiment;

FIG. 2 is a schematic flowchart of an UV disinfection processing method according to an embodiment;

FIG. 3 is a schematic operating diagram of a speed requirement and a power need according to an embodiment;

FIG. 4 is a histogram showing irradiation doses corresponding to different numbers of UV lamps and different travel speeds according to an embodiment; and

FIG. 5 is a schematic diagram showing architecture of another UV disinfection electronic device according to an embodiment.

### DETAILED DESCRIPTION

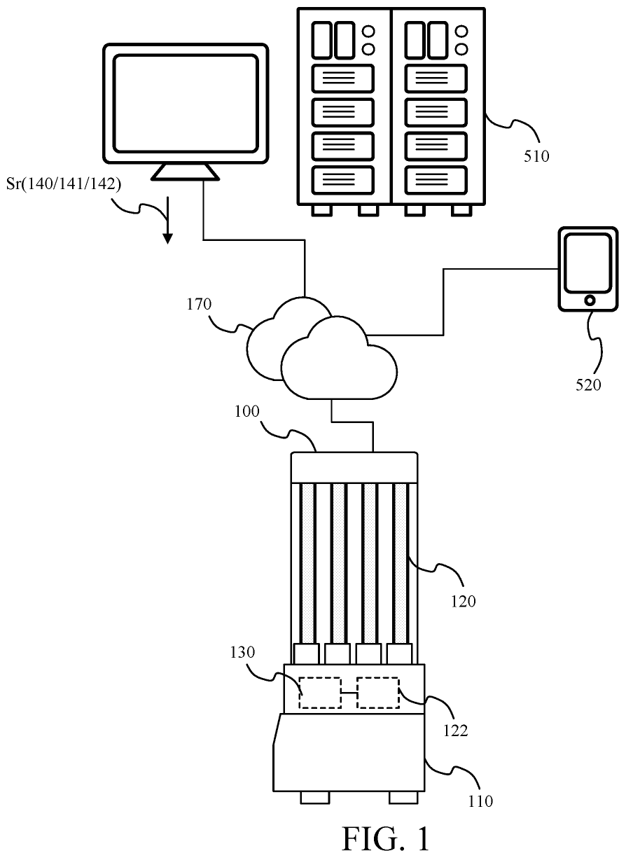

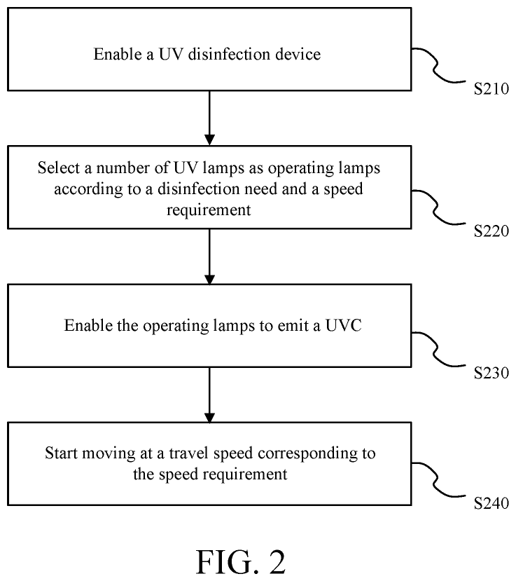

Referring to FIG. 1 and FIG. 2, an UV (Ultraviolet) disinfection electronic device (hereinafter referred to as UV disinfection device 100) includes an autonomous mobile robot 110, an UV lamp group 120 and a microcontroller 130. The microcontroller 130 is electrically connected to the autonomous mobile robot 110 and the UV lamp group 120. In some embodiments, the microcontroller 130 may be implemented by one or more processing units. Each processing unit may be, but not limited to an embedded controller, or an evaluation board (or demo board), a digital signal processor, a central processing unit, a programmable logic controller, or any chip or circuit based on an operation command or operation signal.

The UV lamp group 120 and the microcontroller 130 are disposed on the autonomous mobile robot 110. During the disinfection operation, the microcontroller 130 controls the autonomous mobile robot 110 to move at a travel speed or enables the autonomous mobile robot 110 to stop moving (step S240). Further, the autonomous mobile robot 110 includes a mobile device, a driver and a housing. The microcontroller 130 is disposed in the housing of the autonomous mobile robot 110. The driver is coupled to the microcontroller 130. The microcontroller 130 drives the mobile mechanism such that the mobile mechanism drives the whole UV disinfection device 100 to move. For example, when the autonomous mobile robot 110 is a vehicle using a mobile mechanism such as tires, crawlers or mechanical legs, the mobile mechanism is rotatably embedded in the housing of the autonomous mobile robot 110 and located on a side of the housing adjacent to the ground. Alternatively, when the autonomous mobile robot 110 is a flying device such as an unmanned aerial vehicle (UAV) drone or UV drone, the mobile device such as a propeller or a turbine is installed above, in front of or at a side of the housing.

The UV lamp group 120 includes a plurality of UV lamps 121 and a driving circuit 122. The driving circuit 122 is connected between the UV lamps 121 and the microcontroller 130. In some embodiments, the microcontroller 130 and the driving circuit 122 may be disposed in the housing of the autonomous mobile robot 110, and the UV lamps 121 are assembled on the housing of the autonomous mobile robot 110, that is, exposed outside the autonomous mobile robot 110.

The microcontroller 130 receives a disinfection need, and selects an appropriate number of UV lamps 121 from these UV lamps 121 as operating lamps according to the disinfection need (step S220). During the disinfection operation, the microcontroller 130 controls the driving circuit 122 to drive the operating lamps to emit a UVC according to the disinfection need (step S230). In some embodiments, the operating lamps may emit a UVC after being enabled. It should be understood that the terms “operating lamp” and “UV lamp 121” are only used to distinguish whether they will operate (that is, whether they will emit light or not) during the disinfection operation, and they are only for convenience of explanation, and are not intended to define them as different lamps.

In some embodiments, the aforementioned disinfection need is the minimum irradiation dose to disinfect the target (or spatial field) expected by the disinfection operation. In other words, the irradiation dose provided by the light emitted by the UV lamp group 120 of the UV disinfection device 100 is expected to be relatively greater than or equal to the disinfection need. The formula of the irradiation dose is shown as Formula 1 below.

$$K = I * t , where I = \frac{W}{{M}^{2}} \qquad Formula 1$$

In Formula 1, K is the irradiation dose (in mJ/cm²), I is the irradiation intensity (in mW/cm²), and t is the irradiation time (in s).

In some embodiments, before step S220, the UV disinfection device 100 may receive the disinfection need from an external device (such as a mobile phone or a personal computer). For example, a remote manager (i.e., a user) sets a required disinfection need by means of a monitoring platform 510 or a client 520, and then transmits the disinfection need to the UV disinfection device 100 via a network 170. In some embodiments, the disinfection need may further designate the UV lamp 121 at a specific position as the operating lamp. For example, the disinfection need designates the UV lamps 121 on the same side as the operating lamps. Alternatively, the disinfection need may designate every other UV lamps 121 as the operating lamps. In some embodiments, the UV disinfection device 100 may have a user interface, such that the user can set the required disinfection need in advance and store the disinfection need in the UV disinfection device 100. In an exemplary example, before step S220, the user may directly input a value of the required disinfection need by means of a keyboard on the user interface. In another exemplary example, a plurality of disinfection needs may be stored in the UV disinfection device 100, such that the user can select and set the disinfection need in advance by means of the user interface (not shown) before step S220.

In some embodiments, before step S220, the UV disinfection device 100 may receive a speed requirement 142 from an external device. For example, before step S220, the remote manager sets a required travel speed by means of the monitoring platform 510 or the client 520 so as to generate the speed requirement 142, and then the speed requirement 142 is transmitted to the UV disinfection device 100 for processing via the network 170. In some embodiments the user may also set the required travel speed in advance by using the user interface of the UV disinfection device 100, i.e., input a value of the required travel speed to generate the speed requirement 142 so as to set the travel speed to this value, and then store the travel speed in the UV disinfection device 100. In an exemplary example, the user may directly input the value of the required travel speed by means of the keyboard on the user interface to generate the speed requirement 142 so as to set the travel speed. In another exemplary example, speed options of a plurality of travel speeds may be stored in the UV disinfection device 100, such that the user can select a speed option by means of the user interface before the disinfection operation is started, thereby setting the travel speed in advance.

In some embodiments, the user interface may be a touch screen, a combination of a general display screen and a plurality of physical keys, or a combination of a touch screen and one or more physical keys.

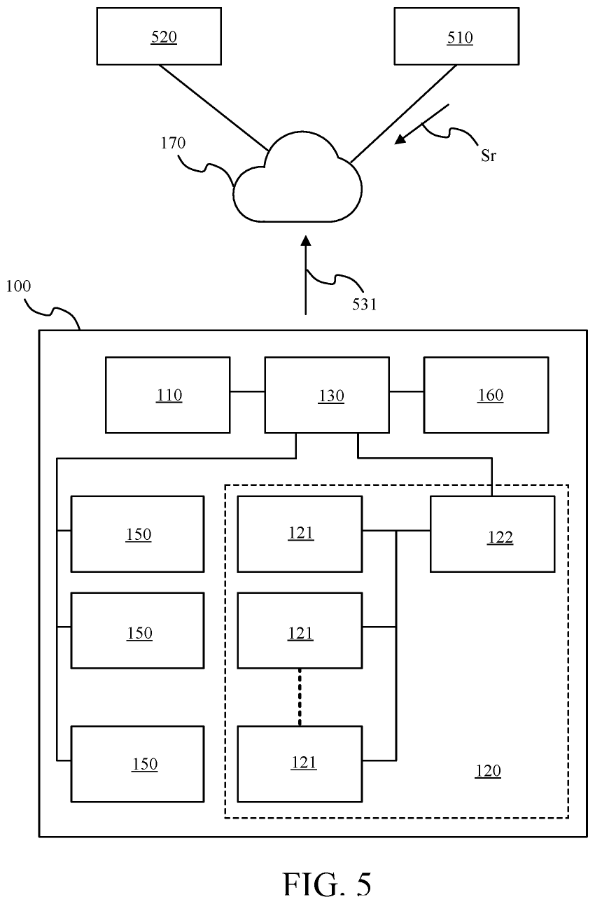

In some embodiments, referring to FIG. 5, the UV disinfection device 100 may further have a communication apparatus 160, and the microcontroller 130 is connected to the communication apparatus 160, such that the microcontroller 130 can be connected to the network 170 via the communication apparatus 160 and further can communicate with the remote monitoring platform 510 or the client 520 via the network 170. In some embodiments, the communication apparatus 160 may include a physical network interface circuit such as a wired or wireless network adapter card.

In some embodiments, the UV disinfection device 100 may further have one or more storage units (not shown) and is configured to store related software/firmware programs, information, data (such as the speed requirement 142 or the disinfection need) and combinations thereof. The microcontroller 130 may have a built-in or external storage unit. In some embodiments, each storage unit may be any type of memory, memory card, or register, and the types of storage units are not limited here.

In some embodiments, the microcontroller 130 enables the UV disinfection device 100 (step S210), drives the autonomous mobile robot 110 according to the speed requirement 142, and enables the autonomous mobile robot 110 to start moving at a travel speed corresponding to the speed requirement 142 (step S240). The user directly sets the speed requirement 142 by means of a control panel of the UV disinfection device 100, or transmits the speed requirement 142 to the UV disinfection device 100 via another device. The disinfection need may also be inputted by means of the control panel of the UV disinfection device 100 or transmitted into the UV disinfection device via another device. Before the autonomous mobile robot 110 starts moving at the travel speed, the microcontroller 130 selects a number of UV lamps 121 as operating lamps according to the disinfection need and the speed requirement 142 (step S220), and enables the operating lamps to emit the UVC (step S230). The number of UV lamps is related to a combination of the speed requirement 142 and the disinfection need. In other words, the microcontroller 130 adjusts the travel speed with respect to brightness of the operating lamps.

If the UV disinfection device 100 moves at a higher travel speed, then it is required to increase the number of UV lamps accordingly, such that the irradiation dose of the operating lamps can meet the disinfection need. In general, the number of UV lamps is selected based on the number of operating lamps that can provide a total irradiation dose closest to the disinfection need, and the irradiation dose is relatively greater than the disinfection need.

In some embodiments, the user may remotely issue an activation command 140 to the UV disinfection device 100, such that the UV disinfection device 100 can be activated to perform the disinfection operation in an unmanned environment. Specifically, when the disinfection operation is to be performed, the microcontroller 130 may receive a disinfection signal Sr via the network 170 so as to enable the UV disinfection device 100 (step S210). The disinfection signal Sr carries the activation command 140. After receiving the disinfection signal Sr, the microcontroller 130 determines the number of UV lamps according to the disinfection need and the speed requirement 142, and selects the number of UV lamps 121 as the operating lamps (step S220). Then, the microcontroller 130 activates the UV disinfection device 100 in response to the activation command 140, enables the autonomous mobile robot 110 to advance at a travel speed corresponding to the speed requirement 142 (step S240), and enables the operating lamps to emit a UVC (step S230). The disinfection signal is a disinfection signal message.

In some embodiments, the disinfection signal Sr may further carry a need requirement 141 of the disinfection need (or the speed requirement 142) for the microcontroller 130 to use or to set a corresponding control parameter (such as the travel speed or the disinfection need). In other embodiments, the disinfection need (or the travel speed) may be set to the need requirement 141 or the speed requirement 142 in advance before step S210, and then the set the disinfection need (or the travel speed) is stored in the storage unit of the UV disinfection device 100. After receiving the disinfection signal Sr, the microcontroller 130 directly reads the current disinfection need (or the travel speed) from the storage unit.

In some embodiments, the UV disinfection device 100 may provide a plurality of travel speeds so as to allow processing of different speed requirements 142. In addition, the UV disinfection device 100 may provide a plurality of disinfection needs so as to allow processing of the need requirements 141 of different disinfection needs.

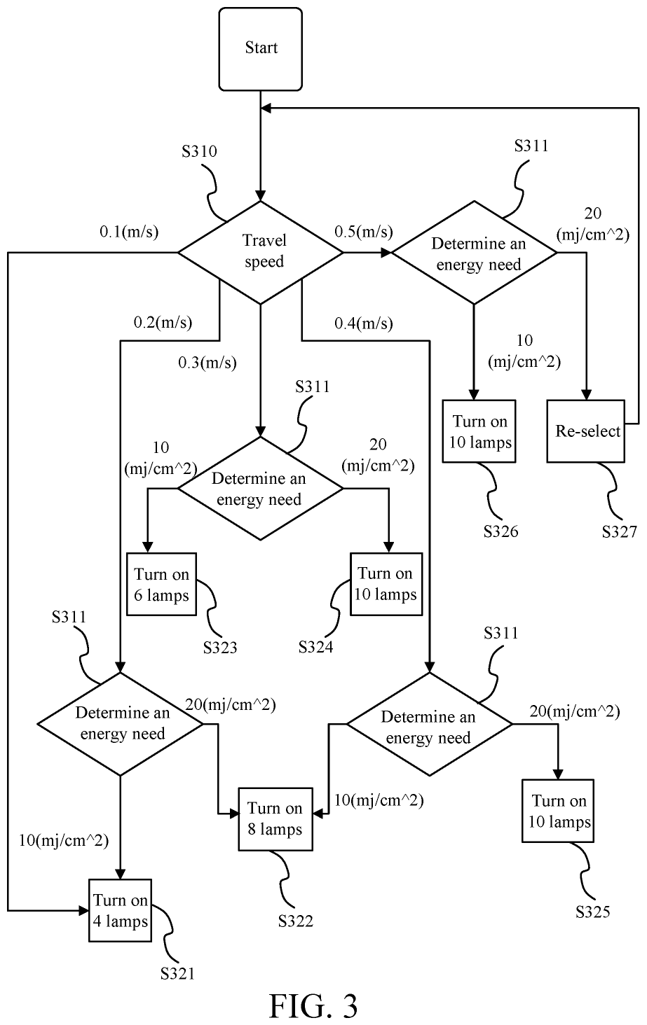

For example, referring to FIG. 1 to FIG. 3, the UV disinfection device 100 having speed options of 5 travel speeds is described below as an example. Here, the 5 travel speeds (step S310) are 0.1 (m/s), 0.2 (m/s), 0.3 (m/s), 0.4 (m/s) and 0.5 (m/s) respectively, and options of the disinfection needs are 10 (mj/cm²) and 20 (mj/cm²) respectively. The UV lamp group 120 has 10 UV lamps 121. The values and numbers of the disinfection needs, the speed requirements 142 and the irradiation doses are only for illustration and are not limited thereto.

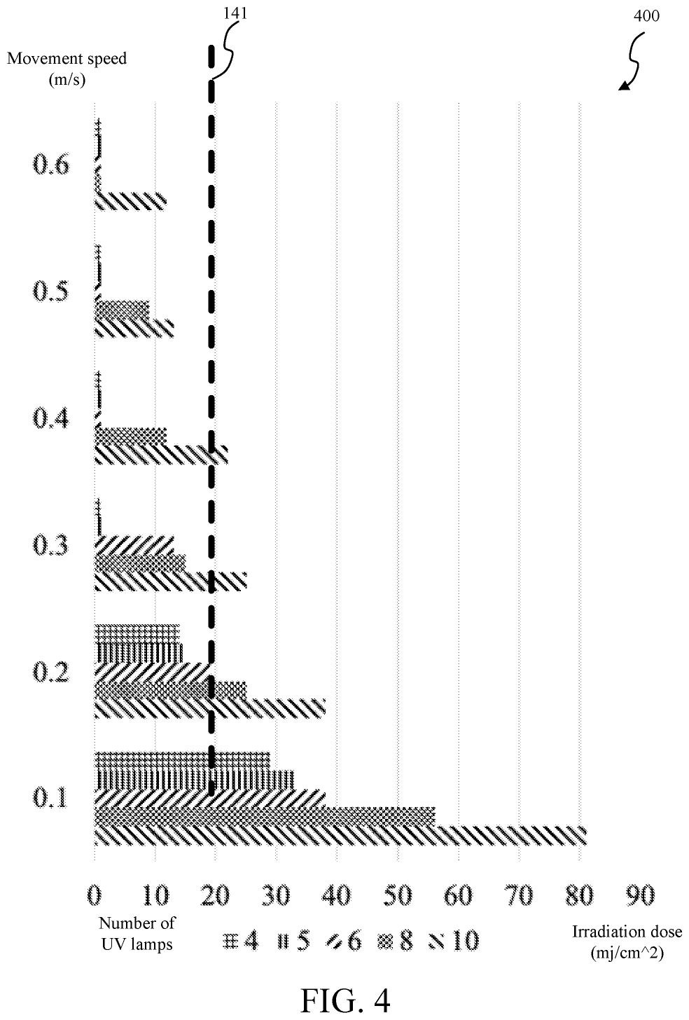

Here, the relationship of the irradiation dose that can be provided by the UV disinfection device 100 under different numbers of UV lamps and different travel speeds is shown in the histogram 400 of FIG. 4. Referring to FIG. 1 to FIG. 4, at each travel speed, the UV disinfection device 100 emits light with different number of operating lamps respectively, and 5 groups of irradiation doses can be measured as the number of UV lamps increases. Here, the different numbers of operating lamps are 10, 8, 6, 5 and 4 respectively. As can be seen from FIG. 4, at the travel speed of 0.5 m/s or above, even if all the UV lamps 121 operate, the disinfection need of 20 (mj/cm²) (indicated by the dashed line in the figure) cannot be achieved. At the travel speeds of 0.3 (m/s) and 0.4 (m/s), only when all the UV lamps 121 operate, the disinfection need of 20 (mj/cm²) can be achieved.

In some embodiments, referring to FIG. 1 to FIG. 3, in step S220, after the UV disinfection device 100 receives the speed requirement 142, the microcontroller 130 first takes the speed requirement 142 or the speed option closest to and greater than or equal to the speed requirement 142 as the travel speed (step S310), then determines the number of UV lamps according to the travel speed corresponding to the speed requirement 142 and the disinfection need (step S311), and determines whether the travel speed corresponding to the speed requirement 142 is greater than or equal to the threshold speed corresponding to the disinfection need. Specifically, the microcontroller 130 obtains the speed requirement 142 from the disinfection signal Sr, and reads the threshold speed corresponding to the disinfection need from the storage unit. Then, the microcontroller 130 confirms the speed requirement 142 according to the threshold speed corresponding to the disinfection need. Different disinfection needs correspond to different threshold speeds. Moreover, the threshold speed is related to not only the disinfection need, but also a total number of the UV lamps 121. In other words, the threshold speed corresponding to each disinfection need depends on the total number of UV lamps 121 of the UV disinfection device 100.

In a case that the speed requirement 142 is relatively smaller than or equal to the threshold speed, the microcontroller 130 sets the travel speed to this speed requirement 142 or a speed option closest to and greater than or equal to the speed requirement 142. The speed option is that the travel speed is set to the speed requirement 142 or closest to and greater than the speed requirement 142. At this time, different speed requirements 142 correspond to different numbers of UV lamps. Therefore, the microcontroller 130 obtains the number of UV lamps based on the speed requirement 142 and the disinfection need by means of a correspondence table or an algorithm (step S321 to step S326).

Conversely, in a case that the speed requirement 142 is relatively greater than the threshold speed corresponding to the disinfection need, the microcontroller 130 takes the threshold speed as the travel speed and designates the number of UV lamps to be the total number of UV lamps 121 (that is, all the UV lamps 121 are used as the operating lamps). In some embodiments, in a case that the speed requirement 142 is relatively greater than or equal to the threshold speed, the microcontroller 130 may generate and transmit a notification message 531 indicating that the speed is too high and needs to be re-selected to the monitoring platform 510 or the client 520, so as to re-acquire the speed requirement 142 (step S327).

For example, it is assumed that the disinfection signal Sr carries the activation command 140 and the speed requirement 142 and the disinfection need of the UV disinfection device 100 has been set. Referring to FIG. 4, when the disinfection need is 20 (mj/cm²), the threshold speed is 0.4 (m/s). When the UV disinfection device 100 selects the travel speed of 0.4 (m/s) based on the speed requirement 142, the irradiation dose of the UV lamp group 120 is greater than 20 (mj/cm²) if all the UV lamps are used as the operating lamps, and the irradiation dose of the UV lamp group 120 is smaller than 20 (mj/cm²) if 8 UV lamps are used as the operating lamps. Therefore, when the number of operating lamps is 10, the disinfection need of 20 (mj/cm²) can be met. As a result, the microcontroller 130 determines the number of UV lamps to be 10 (step S325). Similarly, at the travel speed of 0.4 (m/s), if the disinfection need is 10 (mj/cm²), then the number of UV lamps determined by the microcontroller 130 is 8 (step S322).

When the travel speed of the UV disinfection device 100 is 0.5 (m/s), the irradiation dose of all the 10 operating lamps is still smaller than 20 (mj/cm²), which therefore does not meet the disinfection need of 20 (mj/cm²). As a result, in a case that the speed requirement 142 is equal to 0.5 (m/s), if the disinfection need is set to 20 (mj/cm²), the speed requirement 142 will be regarded as exceeding the threshold speed. Here, the microcontroller 130 may generate and transmit the notification message 531 to the remote manager (i.e., to the monitoring platform 510 or the client 520) (step S327). At this time, the microcontroller 130 may set the travel speed to 0.5 (m/s) and then designate all the UV lamps 121 as the operating lamps, such that the UV disinfection device 100 performs the disinfection operation.

In addition, as can be seen from FIG. 4, at the travel speed of 0.5 (m/s), the irradiation dose of the 10 operating lamps is greater than 10 (mj/cm²). Therefore, the disinfection need of 10 (mj/cm²) is selected, and the microcontroller 130 determines the number of UV lamps as 10 (step S326), thereby driving the autonomous mobile robot 110 to move at the travel speed of 0.5 (m/s) (step S240), and enabling the 10 operating lamps to emit light (step S230).

Similarly, when the speed requirement 142 is 0.65 (m/s), the irradiation dose of the 10 operating lamps is smaller than 20 (mj/cm²), but greater than 10 (mj/cm²). At this time, only the disinfection need of “10 (mj/cm²)” can be selected, so as to comply with the limitation on the selection of the number of UV lamps. In addition, since the speed requirement 142 of 0.65 (m/s) is greater than the maximum speed option of 0.6 (m/s), the microcontroller 130 will take 0.6 (m/s) as the travel speed.

Referring to FIG. 5, in some embodiments, the UV disinfection device 100 may further include a plurality of light sensors 150. The light sensors 150 are connected to the microcontroller 130 and in one-to-one correspondence to the UV lamps 121. In other words, each UV lamp 121 is provided with the corresponding light sensor 150, and each light sensor 150 is disposed on a periphery of the corresponding UV lamp 121. Each light sensor 150 is configured to acquire a photoinduced voltage of the operating lamp at the corresponding position, such that the photoinduced voltage is transmitted to the microcontroller 130. In other words, each light sensor 150 senses the UVC emitted by the corresponding UV lamp 121 and generates an electrical signal of light intensity of the UVC sensed correspondingly (i.e., the photoinduced voltage).

After step S240, the microcontroller 130 receives the photoinduced voltage of each of the operating lamps (i.e., each UV lamp 121 emitting the UVC), and calculates the current irradiation dose according to the photoinduced voltages of the operating lamps and the travel speed corresponding to the speed requirement 142. Next, the microcontroller 130 compares the calculated irradiation dose with the disinfection need so as to determine whether the calculated irradiation dose meets the disinfection need.

If the irradiation dose is relatively smaller than the disinfection need, the microcontroller 130 generates the notification message 531 indicating that an abnormal event (e.g., decay or damage, etc.) has occurred to the operating lamps or that the irradiation dose is insufficient. Here, the microcontroller 130 may store the notification message 531 in the storage unit, so that the user can access the notification message 531 stored in the UV disinfection device 100 by the monitoring platform 510 or the client 520, by connecting a computer apparatus to the UV disinfection device 100, or by directly operating the UV disinfection device 100, so as to confirm the execution effect of the disinfection operation.

Conversely, if the irradiation dose is greater than or equal to the disinfection need, then the microcontroller 130 does not generate the notification message 531, and maintains the current operating state of the UV disinfection device 100, i.e., drives the autonomous mobile robot 110 to move at the travel speed and enables the operating lamps to emit light.

In some embodiments, the UV disinfection device 100 further includes light sensors 150 and a communication apparatus 160. The light sensors 150 and the communication apparatus 160 are connected to the microcontroller 130, and the light sensors 150 are in one-to-one correspondence to the UV lamps 121. Each light sensor 150 is disposed at a periphery of the corresponding UV lamp 121. Each light sensor 150 acquires a photoinduced voltage of the corresponding UV lamp 121, and transmits the photoinduced voltage to the microcontroller 130.

Here, if the currently calculated irradiation dose is relatively smaller than the disinfection need, then the microcontroller 130 generates the notification message 531 indicating that an abnormal event (e.g., decay or damage, etc.) has occurred to the operating lamps or that the irradiation dose is insufficient, and transmits the notification message 531 to the monitoring platform 510 or the client 520 via the communication apparatus 160 (i.e., via the network 170). Conversely, if the irradiation dose is greater than or equal to the disinfection need, then the microcontroller 130 does not generate the notification message 531, and maintains the current operating state of the UV disinfection device 100.

In some embodiments, when the currently calculated irradiation dose is relatively smaller than the disinfection need, the microcontroller 130 may adjust the travel speed or the number of UV lamps in real time, for example, reduce the travel speed of the autonomous mobile robot 110 or increase the number of UV lamps (i.e., increase the number of the UV lamps 121 used as the operating lamps), so that the UV disinfection device 100 moves at a lower travel speed or emits light with more UV lamps 121.

In some embodiments, after step S240, the microcontroller 130 periodically receives and records the photoinduced voltage currently measured by the light sensor 150 corresponding to each UV lamp 121 (or each operating lamp), and compares the photoinduced voltages of the same UV lamp 121 (or the same operating lamp) at adjacent two receptions.

In a case that the photoinduced voltage of any of the UV lamp 121 (or any operating lamp) is reduced (i.e., the currently received photoinduced voltage is smaller than the previously received photoinduced voltage), the microcontroller 130 generates the notification message 531 indicating that an abnormal event (e.g., decay or damage, etc.) has occurred to the operating lamps or that the irradiation dose is insufficient, and transmits the notification message 531 to the monitoring platform 510 or the client 520 via the communication apparatus 160 (i.e., via the network 170). Conversely, in a case that the photoinduced voltages of the same UV lamp 121 (or the same operating lamp) at adjacent two receptions are consistent, the microcontroller 130 does not generate the notification message 531.

In some embodiments, in a case that the photoinduced voltage of any of the UV lamp 121 (or any operating lamp) is reduced, the microcontroller 130 may adjust the travel speed or the number of UV lamps in real time.

In some embodiments, the microcontroller 130 accumulates and records an operation time of each of the UV lamps 121 emitting the UV light (i.e., the sum of durations of light emission of all disinfection operations) by using a timer (not shown). The microcontroller 130 compares the operation time of any of the UV lamp 121 with a threshold time so as to determine whether the operation time of each UV lamp 121 exceeds the threshold time.

If the operation time of any of the UV lamp 121 is greater than or equal to the threshold time, the microcontroller 130 generates a warning notification. Moreover, the microcontroller 130 may transmit the warning notification to the monitoring platform 510 or the client 520 via the communication apparatus 160 (i.e., via the network 170). Conversely, if the operation time of all the UV lamps 121 is smaller than the threshold time, the microcontroller 130 does not generate the warning notification. In some embodiments, the threshold time may be related to a service life of the UV lamp 121. For example, the UV disinfection device 100 may set the threshold time as a preset decay time of the UV lamp 121 in advance.

In some embodiments, the monitoring platform 510 or the client 520 may be a computer apparatus located remotely from the UV disinfection device 100. The computer apparatus may be, for example (but not limited to), a personal computer, a server, a notebook computer, a tablet, a smart phone or the like.

Based on the above, the UV disinfection electronic device and processing method according to any embodiment is applied to disinfection of a spatial field, and can adjust the number of UV lamps 121 emitting the UVC according to the travel speed and the disinfection need, such that the disinfection operation can meet the expected disinfection need. In some embodiments, the UV disinfection electronic device or UV disinfection processing method can automatically adjust the travel speed or the number of UV lamps 121 emitting the UVC when the illumination of any of the UV lamps 121 is decayed. In some embodiments, the UV disinfection electronic device or UV disinfection processing method can notify the remote manager (i.e., the monitoring platform 510 or the client 520) when adjusting the travel speed or the number of UV lamps. In some embodiments, the UV disinfection electronic device or UV disinfection processing method can record the operation time of each of the UV lamps 121, and notify the remote manager when the operation time of any of the UV lamps 121 exceeds the threshold time. In some embodiments, the UV disinfection electronic device or UV disinfection processing method can notify the remote manager when the speed requirement 142 is greater than the threshold time corresponding to the disinfection need, so as to reset the speed requirement and provide other speed requirements 142.

What is claimed is:

1. An Ultraviolet (UV) disinfection electronic device, for an autonomous mobile robot, the UV disinfection electronic device comprising:
an UV lamp group, disposed at the autonomous mobile robot, the UV lamp group including:
a plurality of UV lamps; and
a driving circuit, electrically connected to the plurality of UV lamps, disposed at the autonomous mobile robot and the plurality of UV lamps being driven by the driving circuit; and
a microcontroller, electrically connected to the autonomous mobile robot and the driving circuit, and:
a travel speed of the autonomous mobile robot being controlled by the microcontroller;
the driving circuit being driven by the microcontroller according to a disinfection need, and a number of UV lamps in the plurality of UV lamps being driven by the microcontroller to emit an UV light, the number of UV lamps being related to the travel speed and the disinfection need; and
the travel speed with respect to brightness of the UV light being adjusted by the microcontroller.

2. The UV disinfection electronic device according to claim 1, further comprising a plurality of light sensors, wherein the plurality of light sensors are connected to the microcontroller and respectively correspond to the plurality of UV lamps, each of the light sensors is disposed at a periphery of the corresponding UV lamp, and a photoinduced voltage of the corresponding UV lamp is acquired by each of the plurality of light sensors.

3. The UV disinfection electronic device according to claim 2, wherein the photoinduced voltage of each of the UV lamps emitting the UV light is received by the microcontroller, an irradiation dose according to each of the photoinduced voltages and the travel speed corresponding to a speed requirement is obtained by the microcontroller, and the travel speed is reduced by the microcontroller or the number of UV lamps is increased by the microcontroller according to the irradiation dose and the disinfection need in a case that the irradiation dose is smaller than the disinfection need.

4. The UV disinfection electronic device according to claim 3, further comprising a communication apparatus connected to the microcontroller, wherein in a case that the irradiation dose is smaller than the disinfection need, a notification message is generated by the microcontroller, and the notification message is transmitted to a monitoring platform or a client via the communication apparatus.

5. The UV disinfection electronic device according to claim 2, wherein the photoinduced voltage of each of the UV lamps emitting the UV light is periodically received and recorded by the microcontroller, and the photoinduced voltages of the same UV lamp at adjacent two receptions are compared by the microcontroller, and in a case that the photoinduced voltage of any of the UV lamps is reduced, the travel speed is reduced by the microcontroller or the number of UV lamps is increased by the microcontroller.

6. The UV disinfection electronic device according to claim 5, further comprising a communication apparatus connected to the microcontroller, wherein in a case that the photoinduced voltage of any of the UV lamps is reduced, a notification message is generated by the microcontroller, and the notification message is transmitted to a monitoring platform or a client via the communication apparatus.

7. The UV disinfection electronic device according to claim 1, wherein in a case that a speed requirement is smaller than or equal to a threshold speed corresponding to the disinfection need, a speed option is selected by the microcontroller for setting the travel speed to the speed requirement, or closest to and greater than the speed requirement.

8. The UV disinfection electronic device according to claim 7, further comprising a communication apparatus connected to the microcontroller, wherein in a case that the speed requirement is greater than the threshold speed corresponding to the disinfection need, a notification message is generated by the microcontroller, and the notification message is transmitted to a monitoring platform or a client via the communication apparatus.

9. The UV disinfection electronic device according to claim 7, wherein in a case that the speed requirement is greater than the threshold speed corresponding to the disinfection need, the travel speed is set by the microcontroller to the threshold speed and the number of UV lamps is determined as a total number of the plurality of UV lamps.

10. The UV disinfection electronic device according to claim 1, further comprising a communication apparatus connected to the microcontroller, wherein an operation time of each of the UV lamps emitting the UV light is accumulated by the microcontroller, and in a case that the operation time of any of the UV lamps is greater than or equal to a threshold time, a warning notification is generated by the microcontroller, and the warning notification is transmitted to a monitoring platform or a client via the communication apparatus.

11. The UV disinfection electronic device according to claim 1, further comprising a communication apparatus connected to microcontroller, wherein the microcontroller is further configured to receive a disinfection signal from a monitoring platform or a client via the communication apparatus, the disinfection signal comprises an activation command, and the disinfection signal further comprises a need requirement of the disinfection need, a speed requirement, or both the need requirement of the disinfection need and the speed requirement.

12. An Ultraviolet (UV) disinfection processing method, comprising:
receiving, by an electronic device, a disinfection signal via a network, the disinfection signal comprising an activation command;
determining, by the electronic device, a number of ultraviolet (UV) lamps according to a disinfection need and a speed requirement;
selecting, by the electronic device, the number of UV lamps from a plurality of UV lamps of a UV disinfection device as an operating lamp; and
activating, by the electronic device, the UV disinfection device in response to the activation command, enabling the UV disinfection device to advance at a travel speed corresponding to the speed requirement, and enabling each of the operating lamps to emit an UV light.

13. The UV disinfection processing method according to claim 12, further comprising:
receiving a photoinduced voltage of each of the operating lamps emitting the UV light;
calculating an irradiation dose according to the photoinduced voltage of each of the operating lamps and the travel speed;
comparing the irradiation dose with the disinfection need; and
reducing the travel speed or increasing the number of UV lamps according to the irradiation dose and the disinfection need by the electronic device in a case that the irradiation dose is smaller than the disinfection need.

14. The UV disinfection processing method according to claim 13, further comprising:
generating, by the electronic device, a notification message in a case that the irradiation dose is smaller than the disinfection need; and
transmitting the notification message to a monitoring platform or a client via a network.

15. The UV disinfection processing method according to claim 12, further comprising:
Periodically receiving and recording a photoinduced voltage of each of the operating lamps emitting the UV light;
comparing the photoinduced voltages of the same operating lamp at adjacent two receptions; and
reducing the travel speed or increasing the number of UV lamps in a case that the photoinduced voltage of any of the operating lamps is reduced.

16. The UV disinfection processing method according to claim 15, further comprising:
generating, by the electronic device, a notification message in a case that the photoinduced voltage of any of the operating lamps is reduced; and
transmitting the notification message to a monitoring platform or a client via a network.

17. The UV disinfection processing method according to claim 12, further comprising:
confirming the speed requirement according to a threshold speed corresponding to the disinfection need, different disinfection needs corresponding to different threshold speeds;
generating, by the electronic device, a notification message in a case that the speed requirement is greater than the threshold speed; and
transmitting the notification message to a monitoring platform or a client via a network.

18. The UV disinfection processing method according to claim 17, further comprising:
selecting, by the electronic device, a speed option in a case that the speed requirement is relatively than or equal to the threshold speed, the speed option is that the travel speed is set to the speed requirement or closest to and greater than the speed requirement.

19. The UV disinfection processing method according to claim 17, wherein in a case that the speed requirement is greater than the threshold speed, in the step of determining the number of UV lamps according to the disinfection need and the speed requirement, the number of UV lamps determined is a total number of the plurality of UV lamps, and the processing method further comprises: setting the travel speed to the threshold speed.

20. The UV disinfection processing method according to claim 12, further comprising:
accumulating an operation time of each of the UV lamps emitting the UV light;
comparing the operation time of each of the UV lamps with a threshold time;
generating, by the electronic device, a warning notification in a case that the operation time of any of the UV lamps is greater than or equal to the threshold time; and
transmitting the warning notification to a monitoring platform or a client via a network, wherein the disinfection signal further comprises a need requirement of the disinfection need, a speed requirement, or both the need requirement of the disinfection need and the speed requirement.
